(19) United States
(12) Patent Application Publication
(10) Pub. No.: US 2026/0083789 A1
(43) Pub. Date: Mar. 26, 2026

(54) FORMULATION FOR STIMULATING HAIR GROWTH AND IMPROVING QUALITY AND METHOD OF PREPARATION OF THE SAME

(71) Applicant: ARJUNA NATURAL PRIVATE LIMITED, Kerala (IN)

(72) Inventor: Benny ANTONY, Kerala (IN)

(73) Assignee: ARJUNA NATURAL PRIVATE LIMITED, Kerala (IN)

(21) Appl. No.: 19/111,006

(22) PCT Filed: Sep. 14, 2023
(86) PCT No.: PCT/IB2023/059131

§ 371 Date: Mar. 12, 2025

(30) Foreign Application Priority Data
Sep. 17, 2022 (IN) 202241053235
May 11, 2023 (IN) 202341033357

(51) Int. Cl. A61K 36/21; A61K 9/00; A61K 47/06; A61K 47/10; A61K 47/12; A61K 47/22; A61K 47/44; A61P 17/14

(52) U.S. Cl. CPC A61K 36/21; A61K 9/0019; A61K 47/06; A61K 47/10; A61K 47/12; A61K 47/22; A61K 47/44; A61P 17/14

(57) ABSTRACT

The present invention relates to a formulation for stimulating hair growth and improving quality and the active ingredient derived is from plant sources, especially comprising 10-20% nitrate enriched extract of Amaranth. In addition, said formulation is having one or more ingredients selected from liquid paraffin, mango butter, bees wax, cetyl alcohol, glycerol, sodium benzoate, potassium Sorbate. It also discloses a method of preparation of said formulation, its application as well as its use for stimulating hair growth and improving hair quality. It is found that said formulation has high wound healing potential at lower concentration by Increasing the rate of cell proliferation. It increases not only the Hair growth rate but also increases total hair count and density (n/cm²) of Terminal hair, Vellus hair and Anagen hair. Applying said formulation on the scalp helps in stimulating hair growth and improving the quality of hair.

### FILED OF INVENTION

The present invention relates to a formulation for stimulating hair growth and improving hair quality. The formulation of the present invention comprises active ingredients from plant sources such as green leafy vegetables such as Amaranth, spinach, lettuce, as well as fennel, rocket, radishes, Chinese cabbage, and parsley containing nitrate, more especially the extract of Amaranth. The present invention also relates to methods of preparation of said formulation, its application as well as its use for stimulating hair growth and improving hair quality.

### BACKGROUND OF INVENTION

Hair is integral to our body image and can have a profound influence on our self-esteem and self-confidence. The growth and loss of hair may seem like a simple process, but the hair growth cycle is actually composed of four distinct phases. The first three phases—anagen, catagen, and telogen—cover the growth and maturation of hair and the activity of the hair follicles that produce individual hairs. During the final, or exogen phase, “old” hair sheds, though usually, a new hair is getting ready to take its place. Each phase has its own timeline, which can be affected by age, nutrition, and overall health.

The stages of hair growth begin with the anagen phase. It's the longest phase, lasting about 3 to 5 years. During the anagen phase, hair follicles are pushing out hairs that will continue to grow until they're cut or until they reach the end of their lifespan and fall out.

The catagen phase starts when the anagen phase ends, and tends to last about 10 days or so. During this chapter, hair follicles shrink and hair growth slows. The hair also separates from the bottom of the hair follicle, yet remains in place during its final days of growing.

The telogen phase typically lasts around 3 months. An estimated 10 to 15 percent of our scalp hairs are in this phase. Hairs don't grow during the telogen phase, but they don't usually fall out either. The telogen phase is also when new hairs start to form in follicles that have just released hairs during the catagen phase.

The exogen phase is essentially an extension or a part of the telogen stage of hair growth. During the exogen phase, hair is shed from the scalp, often helped along by washing and brushing. Losing 50 to 100 hairs per day during the exogen phase is normal.

Hair loss is a common problem experienced by many humans as well as many animals. There are many types of hair loss, also called alopecia. Alopecia is a chronic dermatological disorder in which people lose some or all of the hair on their head and sometimes on their body as well. Major factors that may influence hair loss are hormonal changes, genes, stress, illness, childbirth, drugs, burns, injuries, autoimmune disease, cosmetic procedures and diet. Hereditary androgenic alopecia is the commonest form of alopecia: it is manifested by a decrease in hair volume, or even baldness, and affects up to about 70% of men. Acute alopecia may be associated with treatment by chemotherapy, stress, severe malnutrition, iron deficiency, hormonal disorders, AIDS, or acute irradiation.

Androgenetic alopecia (AGA), also known as male-pattern hair loss (MPHL), is a genetically determined progressive condition in which terminal hair is gradually replaced by vellus hair. The prevalence in men rises with age, but the ages at which symptoms appear and the rate at which they progress vary. The temples, vertex scalp, and mid-frontal scalp are the three sections of the scalp most impacted. The process is meticulously structured within these areas. In females, pattern hair loss (FPHL) is distinguished by its diffuse crown thinning and intact frontal hairline.

Alopecia areata often starts suddenly and causes patchy hair loss in children and young adults. This condition may result in complete baldness (alopecia totalis). Alopecia universalis causes all body hair to fall out, including the eyebrows, eyelashes, and pubic hair. Trichotillomania, seen most frequently in children, is a psychological disorder in which a person pulls out one's own hair. Telogen effluvium is temporary hair thinning over the scalp that occurs because of changes in the growth cycle of hair. A large number of hairs enters the resting phase at the same time, causing hair shedding and subsequent thinning. Scarring alopecias result in permanent loss of hair. Inflammatory skin conditions (e.g., cellulitis, folliculitis, acne), and other skin disorders (such as some forms of lupus and lichen planus) often result in scars that destroy the ability of the hair to regenerate.

Literature indicates the involvement of autocrine and paracrine factors along with signaling pathways in the cross-talk between the dermal papillae and the hair follicle stem cells. Dihydro testosterone (DHT) binding to the AR is the primary cause of androgen-dependent processes. Significant progress has been made in understanding the key elements of androgen metabolism involved. Various studies in patients with androgen insensitivity syndromes and 5α-reductase type 2 deficiencies have suggested activation of follicular androgen receptors by dihydrotestosterone induces androgenic alopecia. The availability of weak androgens, their conversion to more strong androgens via the action of 5α-reductase, low enzymatic activity of androgen inactivating enzymes, and a large number of functionally active AR is all required for DHT-dependent cell functions. Thus, DHT levels are high in the prone scalp, and AR expression is enhanced. The conversion of testosterone to DHT in the dermal papilla is important, and androgen-regulated substances derived from dermal papilla cells are thought to influence the proliferation of other hair follicle components. The proportional contributions of locally produced dihydrotestosterone and systemically produced dihydrotestosterone to the balding process are unknown. Thus, it can be said that the putative component in the complicated aetiology of AGA is prolonged microscopic follicular inflammation with connective tissue remodeling, which eventually results in irreversible hair loss (Scheme 1).

One known treatment for alopecia is hair transplantation. Another treatment is drug therapy. The only products sanctioned by the US FDA for hair loss treatment are oral finasteride, a competitive inhibitor of type 2, 5-α-reductase, and topical minoxidil, an adenosine-triphosphate-sensitive potassium channel opener that has been shown to stimulate the production of vascular endothelial growth factor in cultured dermal papilla cells. Minoxidil is available as topical preparations which may be used for men and women. Minoxidil is a piperidino-pyrimidine derivative (2,4-diamino-6 piperidino-pyrimidine-3-oxide) that acts as a prodrug, requiring conversion to its active metabolite, minoxidil sulphate (MS), in order to exert its pharmacological effects. The active metabolite is responsible for minoxidil's vascular and follicular actions. It usually needs to be used for several months before any effect is seen. The balding process will usually resume if treatment with minoxidil is stopped. The re-grown hair may fall out three to four months after treatment is stopped. One of the severe effects that minoxidil associated with is hypertrichosis, especially on the face in women. Many patients are hesitant to use it because their hair appears to be dry, dull, and stiff at times. There are reports where patients have complaint about palpitations, irritation or burning of eyes, and weight gain. Topical minoxidil is also reported to have an adverse impact on the cardiovascular system.

Finasteride has also demonstrated some efficacy in treating hair loss. Finasteride is a 5a-reductase inhibitor, which functions by blocking the conversion of testosterone to the active 5a-dihydrotestosterone (DHT) form, elevated levels of which have been linked to hair loss. Studies indicate effectiveness in approximately 50% of patients, with side effects including erectile dysfunction and gynecomastia being reported. Furthermore, finasteride is not indicated for use in women of childbearing age as it can cause birth defects in unborn babies.

Pharmacological approaches have their own set of drawbacks. As a result, recent medical attention on AGA has been focused on the discovery of new and safer treatments, which are frequently provided by natural therapies. Literature indicates the use of herbal medicine to treat disorders, like alopecia. However, they all lack mechanistic explanations for their action. There is literature on the activities of seven common botanicals (Panax ginseng C.A. Mey., Malus pumila Mill cultivar Annurca, Coffea arabica, Allium sativum L., Camellia sinensis (L.) Kuntze, Rosmarinum officinalis L., Capsicum annum L.) that are thought to reduce the rate of hair loss or stimulate new hair growth. Though their positive effects are largely discussed in the literature, they too suffer from limitations such as the known gap between different experimental strategies—in vitro, ex vivo, in vivo, and clinical trials—which makes integrative mechanistic explanations difficult. As a result, alternative treatments such as physical or cosmetic treatments, supplements, and the use of herbal extracts are required.

Traditional food plants are consumed by a large portion of the population in developing countries such as India. It is estimated that approximately 30,000 plant species worldwide are edible, with only 7000 having been used as food. Natural compounds extracted from plants, such as storage lipids, fragrances, essential oils, flavonoids and polyphenols, have been extensively researched for their food value and are used as precursors by the cosmetics and pharmaceutical industries.

In the present invention, a topical formulation comprising nitrate enriched extract of Amaranth is used for stimulating hair growth and improving hair quality. Amaranth is an excellent source of protein, dietary fiber, nitrates and minerals. The nitrates convert into nitrites and then to nitric oxide inside the body. Nitric oxide has been known to be a potent vasodilator. The increased blood flow to the hair cells is hypothesized to promote follicle growth and regeneration. It also contains a critical amino acid Lysine which is need for the body to create healthy and stronger hair with strong roots.

The present invention provides the in vitro and in vivo effect of nitrate enriched extract of Amaranth on DHT induced hair loss and elucidate its possible mechanism of action.

### SUMMARY OF THE INVENTION

We disclose a formulation for stimulating hair growth and improving quality and the active ingredient is derived from plant sources, especially nitrate rich extract. Plant sources used in the formulation are selected from Amaranth, spinach, lettuce, as well as fennel, rocket, radishes, Chinese cabbage, and parsley. Other sources are Beet root, radishes, turnips, watercress, celery etc. In one preferred embodiment, the nitrate rich extract derived from Amaranth is used in said formulation. In addition to nitrate enriched extract of Amaranth in the formulation, it can have one or more ingredients selected from liquid paraffin, mango butter, bees wax, cetyl alcohol, glycerol, Sodium Benzoate, Potassium Sorbate along with water.

According to a preferred embodiment under the invention the formulation is comprising of 10-20% nitrate enriched extract of Amaranth. In another embodiment formulation comprises 10-20% nitrate enriched extract, 20-30% of liquid paraffin, 20-30% mango butter, 10-15% bees wax, 5-10% cetyl alcohol and 15-25% glycerol, preferably said formulation consists of 15% nitrate enriched extract of Amaranth, 20% liquid paraffin, 25% mango butter, 14% bees wax, 6% cetyl alcohol and 20% glycerol.

According to another embodiment under the invention said formulation consisting of the formulation of the invention comprises 10-20% nitrate enriched extract, 20-30% liquid paraffin, 20-30% bees wax, 5-10% cetyl alcohol, 15-25% glycerol, 0.1-3% sodium benzoate, 0.1-3% potassium Sorbate and 1-10% water, preferably consisting of 15% nitrate enriched extract of Amaranth, 30% liquid paraffin, 23% bees wax, 6% cetyl alcohol, 20% glycerol, 0.5% Sodium Benzoate, 0.5% Potassium Sorbate and 5% water.

It is found that said formulation is having high wound healing potential at lower concentration by Increasing the rate of cell proliferation. It increases not only the Hair growth rate but also increases total hair count and density (n/cm²). As part of efficacy evaluation, it was applied on the temples, vertex scalp, and or mid-frontal scalp of clinically diagnosed male pattern hair loss (MPHL) at a dose of 2 g once daily at night for 90 days. The primary outcome was a mean change in the density, diameter and count of Terminal hair. The secondary outcome was a mean change in the density, diameter and count of vellus, anagen, telogen, pilary index, terminal to vellus ratio, and anagen to telogen ratio.

On application said formulation is found increases VEGF mRNA expressions which indicates the formation of new blood vessels at the site of action/wound site. It also increases Pilary index which is the percentage of anagen hairs multiplied by its diameter.

A method of preparation of the formulation as claimed in claim 1 for stimulating hair growth and improving quality comprising of:
combining bees wax and liquid paraffin each in an amount ranging from 20-30% and melting in a water bath to form an oil phase;
combining Cetyl alcohol and liquid paraffin each in an amount ranging from 5-10% and melting to form an oil phase;
dissolving Sodium benzoate and Potassium sorbate each in an amount ranging from 0.1-3% in water separately to form water phase;
Nitrate enriched extract of Amaranth and glycerol in an amount ranging from 10-20% and 15-25% respectively mixed with stirring to form water phase; and
adding the resultant water phase and oil phase together while stirring resulting into the formulation for stimulating hair growth and improving quality.

### BRIEF DESCRIPTION OF FIGURES AND DRAWINGS

These and other features, aspects and advantages of the present invention will become better understood when the detailed description is read with reference to the accompanying drawing.

FIGS. 1(a) and (b) are representative of the MTT Assay which revealed that nitrate enriched extract of Amaranth had no side effects on the human dermal fibroblasts.

FIGS. 1(c), (d) and (e) depict a rise in VEGF expression for A1 as well as A2 for both Minoxidil as well as nitrate enriched extract of Amaranth.

FIG. 2 represents total hair count after applying topical formulation and placebo.

FIG. 3 represents total hair density after applying topical formulation and placebo.

FIG. 4 represents total hair length after applying topical formulation and placebo.

FIG. 5 represents terminal hair count after applying topical formulation and placebo.

FIG. 6 represents terminal hair density after applying topical formulation and placebo.

FIG. 7 represents Vellus hair count after applying topical formulation and placebo.

FIG. 8 represents Vellus hair density after applying topical formulation and placebo.

FIG. 9 represents Anagen hair count after applying topical formulation and placebo.

FIG. 10 represents Anagen hair density after applying topical formulation and placebo.

FIG. 11 represents Pilary Index after applying topical formulation and placebo.

FIG. 12 represents Anagen/telogen ratio after applying topical formulation and placebo.

### DETAILED DESCRIPTION OF THE INVENTION

The present invention discloses formulation for stimulating hair growth and improving hair quality. Each novel feature is disclosed through embodiment. Through these embodiments, a formulation, a process of preparation of said formulation as well as a method of use for stimulating hair growth and improving hair quality are disclosed here. The embodiments have to be understood in its broadest sense. The illustrations also disclose the efficacy of said formulations over known counterparts through various in vivo and in vitro studies. The illustrations for the invention are meant for better understanding of the invention for a person skilled in the art, and do not intend to narrow the scope of any subject matter claimed.

As used herein, the term “hair” refers to the protein filament that grows from follicles found in the dermis and includes scalp, head, facial, and/or body hair, eyelashes, brows, mustache, beard, ear, nasal, chest, pubic, auxiliary, fur, and the like.

By “enhancing hair growth” or “stimulating hair growth” or “inducing hair growth” or “promoting hair growth” is meant the earlier induction of growth of a new hair cycle, and/or prolonging the active growth phase (anagen) of the hair cycle, and/or increasing the growth rate of the hair, and/or increasing the width of the hair shaft, including, but not limited to, the induction of the growth of hair and making it more visible to the eye.

As used herein “improving hair quality” means increasing the diameter of the hair shaft and/or enhancing the visual attributes of the hair like hair volume, hair shine, and hair thickness, and/or affecting the characteristics of the hair shaft, and/or hair cuticles, including, but not limited to, creating a smoother look or feel, and/or increase in shine.

Improved vasodilation and blood flow to the scalp provides more oxygen and nutrients to the dermal papillae that can stimulate the hair follicles to grow new shafts and prolong the anagen growth phase. Though anagen duration is genetically determined, adequate nourishment from circulation supports mitosis and follicle cell proliferation. While other factors beyond nutrition regulate anagen-to-telogen transition, diminished blood supply is detrimental, especially in androgen mediated vasoconstriction by activating endothelial production of vasoconstrictors.

The regulation of the hair cycle takes place at the pilo-sebaceous unit, which includes the sebaceous gland, hair follicle, and hair shaft. Endothelin-1 is a potent vasoconstrictor produced from vascular endothelial cells in response to high testosterone level acting on androgen receptors. This vasoconstriction might be a reason inducing male pattern hair loss by restricting nutrients to dermal papillae and causing miniaturization in androgenic alopecia.

Nitric oxide (NO) is a potent vasodilator and signaling molecule in the body. Endothelial production of nitric oxide triggers a cascade of cellular signaling pathways that stimulate hair growth. This signaling activates anti-inflammatory pathways creating optimal microenvironment for hair regeneration as chronic inflammation damages follicles and leads to progressive hair loss. NO increases cellular metabolism and energy production in hair follicles. This metabolic stimulation provides more of the biological fuel and building blocks necessary for hair follicle cell proliferation and hair shaft production. So, the nitric oxide always balances the vasoconstrictive effects of ET-1 in the blood vessels surrounding the dermal papillae.

In one embodiment, the present disclosure relates to a method for enhancing hair growth in a subject. The method comprises applying a formulation comprising nitrate enriched extract to the subject.

In one embodiment, the present disclosure relates to a method for improving hair quality on a subject. The method comprises applying a formulation comprising nitrate enriched extract to the subject.

In one embodiment, the present disclosure relates to a method for preventing and/or treating hair loss on a subject. The method comprises applying a formulation comprising nitrate enriched extract to the subject.

One embodiment of the present invention provides a formulation e.g., a topical formulation, comprising nitrate enriched extract and excipients, effective to prevent hair loss and/or promote hair growth and/or enhance hair growth. By “topical formulation” is meant that the formulation is capable of being applied externally to the dermis of a mammal. For example, the formulation may be useful for administration to the skin of a male subject who is susceptible to hair loss or at risk of hair loss, such as a subject suffering from or at risk of developing male-pattern baldness. In another example, the formulation of the invention is useful for administering skin of a subject suffering from a disease or condition associated with hair loss e.g., alopecia, especially acute alopecia or androgenic alopecia.

In one embodiment, the topical formulations of the invention are useful for the stimulation of hair growth in subjects suffering from or having a predisposition to developing androgenic alopecia or male pattern baldness.

In one embodiment, the topical formulations of the invention are useful for improving the quality of hair in subjects.

One embodiment of the present invention provides a method of treatment or prevention of hair loss, said method comprising administering to a subject in need thereof e.g., a subject suffering from alopecia or having a tendency to develop alopecia, a formulation, e.g., a topical formulation, comprising nitrate enriched extract, and excipients, effective to prevent hair loss and/or promote hair growth and/or enhance hair growth on the subject. The subject will generally be a mammal such as a human.

One embodiment of the present invention provides a method of improving hair quality, said method comprising administering to a subject in need thereof, a formulation, e.g., a topical formulation, comprising nitrate enriched extract, and excipients, effective to improve the quality of hair on the subject. The subject will generally be a mammal such as a human.

Nitrate rich extract used for making the formulation is obtained from plant sources such as green leafy vegetables such as Amaranth, spinach, lettuce, as well as fennel, rocket, radishes, Chinese cabbage, and parsley. Other sources are Beet root, radishes, turnips, watercress, celery etc. In one preferred embodiment, the nitrate plant source is Amaranth.

Excipients will typically be included in the dosage form e.g., to improve solubility and/or bioadhesion. Suitable excipients include solvents, co-solvents, emulsifiers, plasticizers, surfactants, thickeners, pH modifiers, emollients, antioxidants, and chelating agents, wetting agents, and water absorbing agents. Formulations may also include one or more additives, for example, dyes, colored pigments, pearlescent agents, deodorizers, and odour maskers.

Suitable excipients include but not limited to liquid paraffin, petrolatum, cera microcristallina, microcrystalline wax, ozokerite, ceresine isoparaffin, paraffin, synthetic wax, mango butter, shea butter, cocoa butter, Avocado butter, rice bran wax, Soya wax, Lauric acid, Olive oil, Cetyl Alcohol, glycerol, triethylene glycol, tripropylene glycol, propylene glycol.

The formulation can be adapted for administration by any appropriate route, for example by the oral, topical, or parenteral route.

The formulation may be applied topically and may be in the form of a cream, a lotion, an ointment, a gel, a liquid, or any other topical form.

Pharmaceutical formulations may be presented in unit-dose forms containing a predetermined amount of active agent.

In one embodiment, the formulation of the invention comprises 10-20% nitrate enriched extract of Amaranth, 15-25% liquid paraffin, 20-30% mango butter, 10-15% bees wax, 5-10% cetyl alcohol and 15-25% glycerol.

In one preferred embodiment, the formulation of the invention comprises 15% nitrate enriched extract of Amaranth, 20% liquid paraffin, 25% mango butter, 14% bees wax, 6% cetyl alcohol and 20% glycerol.

In another embodiment, the formulation of the invention comprises 10-20% nitrate enriched extract, 20-35% liquid paraffin, 20-30% bees wax, 5-10% cetyl alcohol, 15-25% glycerol, 0.1-3% sodium benzoate, 0.1-3% potassium Sorbate and 1-10% water.

In the preferred embodiment, the formulation of the invention comprises 15% nitrate enriched extract of Amaranth, 30% liquid paraffin, 23% bees wax, 6% cetyl alcohol, 20% glycerol, 0.5% Sodium Benzoate, 0.5% Potassium Sorbate and 5% water.

In one embodiment, the method of making the topical formulation is disclosed. Bees wax and liquid paraffin in equal amounts are combined and melted in a water bath to form an oil phase. Cetyl alcohol and liquid paraffin are taken, combined, and melted to form an oil phase. Sodium benzoate and Potassium sorbate are dissolved in water separately to form water phase. Nitrate enriched extract of Amaranth and glycerol are mixed with stirring to form water phase. All water phases are added to oil phases by continuous stirring.

Efficacy of the nitrate enriched extract of Amaranth is studied in vitro using Human dermal fibroblasts (HDFa) cells. MTT Assay revealed that nitrate enriched extract of Amaranth had no side effects on the human dermal fibroblasts (HDFs). In fact, under the influence of nitrate enriched extract of Amaranth at 48 hours, HDFs exhibited ˜120 % cell proliferation. Cell cytotoxicity, however is observed at 72 hours time interval.

Further, in contrast to nitrate enriched extract of Amaranth, Minoxidil displayed increased cell proliferation at lower concentrations. Nonetheless, in comparison with nitrate enriched extract of Amaranth which showed its efficacy within 24 hours, Minoxidil took 48 hours to increase the rate of cell proliferation and peaked at 72 hours.

It is also evident from the MTT results that nitrate enriched extract of Amaranth has a faster mode of action as compared to Minoxidil. This faster mode of action could be ascribed to the water-soluble nature of nitrate enriched extract of Amaranth, while Minoxidil is an oil-based formulation.

Vascular endothelial growth factor (VEGF) is a potent angiogenic factor and was first described as an essential growth factor for vascular endothelial cells. VEGF mRNA expressions are examined by quantitative real-time PCR. The relative mRNA expression is greater in case of nitrate enriched extract of Amaranth with A2 at 48 hours time interval being the maximum. This increased relative mRNA expression also indicates the formation of new blood vessels at the site of action/wound site. It is also important to note that the mRNA expression for Minoxidil at M1 and M2.

Efficacy of the topical formulation is studied on male pattern hair loss. Males with clinically diagnosed male pattern hair loss are selected for the study. Topical formulation of nitrate enriched extract of Amaranth is applied on vertex at a dose of 2 g once daily at night for 90 days. The top of the back of the head is often called the vertex and represents the original midline location of the posterior fontanelle or baby soft spot. Between these borders lines lies the occipital convexity. Mean change from baseline in the Terminal hair (Non-Vellus hair) density (n/cm²), hair-shaft diameter (mm), and hair growth rate (mm/day) is measured from day 3 to day 93. Mean change from baseline in the Target area total hair count, thickness (mm) and length (mm), Mean change from baseline in the Pilary Index, Mean change from baseline in the vellus hair count, density and percentage, Mean change from baseline in the percentage & density of Anagen hair, Mean change from baseline in the percentage & density of Telogen hair, Mean change from baseline in the Terminal to Vellus hair ratio, Mean change from baseline in the anagen to telogen hair ratio, Mean change from baseline in the dermatology life quality index (DLQI), Change from baseline in PGIC are measured from day 3 to day 93.

The hair density is calculated as the number of hairs per square centimetre. Terminal hair density is the number of terminal hairs per square centimetre. Terminal hair is thicker than 40 μm and this value is used by the software to identify terminal hairs in images. Vellus hair density (n/cm²) is the number of vellus hairs per square centimetre. By definition a vellus hair is thinner than 40 μm. Anagen hair density (n/cm²) is the number of anagen hairs per square centimetre. Anagen hair grows at approximately 0.3 mm/day. Telogen hair density (n/cm²) is the number of telogen hairs per square centimetre. Telogen hairs will not grow. Pilary Index is the percentage of anagen hairs multiplied by its diameter.

The total hair count and hair density increased significantly by 23% from baseline while placebo had 11% change at day 90. The hair growth rate (μm/day) is 380 μm/day for test and 310 μm/day for placebo averaged across a study period of 90 days. The terminal hair count and density increased significantly by 28% from baseline while placebo had 15% change. The vellus hair count and density increased by 12% in the test group whereas there is only 3% change in the placebo.

In this study the anagen hair density increased by 54% compared to placebo. The anagen by telogen ratio showed significant increase of 155% whereas placebo showed only 21% change.

The pilary index (PI) is a measure of the hair growth cycle in androgenetic alopecia. A lower pilary index indicates a more advanced stage of Male Pattern Hair Loss (MPHL).

In the present study the increase in PI (23%) was noticeable as early as after 45 days of application of the test product while placebo had negligible change of 0.11%. At the end of the 90 day study there was a significant increase of 25% change while placebo had 7% change.

The Dermatology Life Quality Index (DLQI) is a simple, self-administered and user-friendly validated questionnaire. 10 questions are asked pertaining to symptoms and feelings, daily activities, leisure, work and school, personal relationships and treatment.

The scoring of each answer is as follows: The DLQI is calculated by adding the score of each question. The maximum score is 30 and the minimum is 0. The higher the score, the more quality of life is impaired.

### Meaning of DLQI Scores

0-1—no effect at all on patient's life
2-5—small effect on patient's life
6-10—moderate effect on patient's life
11-20—very large effect on patient's life
21-30—extremely large effect on patient's life

### Subjects Global Improvement of Change

PGIC is a 7-point scale depicting a patient's rating of overall improvement. Patients rate their change as “very much improved,” “much improved,” “minimally improved,” “no change,” “minimally worse,” “much worse,” or “very much worse.”

### Examples

### Method of Preparation of the Topical Formulation

230 g Bees wax and 230 g liquid paraffin were combined and melted in a water bath to form an oil phase. 60 g Cetyl alcohol and 70 g liquid paraffin were taken, combined, and melted to form an oil phase. 5 g Sodium benzoate and 5 g Potassium sorbate were dissolved in water separately to form water phase. 150 g nitrate enriched extract of Amaranth and 200 g glycerol were mixed with stirring to form water phase. All water phases are added to oil phases by continuous stirring.

### In Vitro Study for the Efficacy of the Nitrate Enriched Extract of Amaranth Using Human Dermal Fibroblasts (HDFa) Cells.

### Cell Culture

Human dermal fibroblasts (HDFa) cells were maintained in DMEM high glucose media (Himedia) with 10% FBS and 40 units/ml of penicillin & 40 μg/mL of streptomycin (Thermo Fisher) in 37° C. humidified chamber with 5% CO2. Cells were passaged after every 2-3 days using trypsin-EDTA.

### MTT Assay

Cells were trypsinized, centrifuged and resuspended in culture medium. Cells and then counted by trypan blue method using haemocytometer and plated at 5000 cells per 50 μl into wells of a 96 well microtiter plate. The cells were then incubated overnight under conditions appropriate for the cell line for adherence. Following this, treatment was given to cells with the different concentrations of nitrate enriched extract of Amaranth or Minoxidil for varied number of time period (24 hours, 48 hours and 72 hours). 10 μl MTT reagent was then added to each well, after respective time period followed by incubation for 4 hours. After 4 hours, when the purple precipitate was clearly visible under the microscope, supernatant was removed carefully by pipette and 100 μl of solubilization solution (DMSO) was added to all wells. The plate was shaken softly for 20 minutes in the dark at room temperature. Then, the absorbance was taken of each well, including the blanks, at 570 nm with reference wavelength at 630 nm in a microtiter plate reader. The average values were determined from triplicate readings and subtracted the average value for the blank. Absorbance of test was normalized with the control wells to determine the cell viability which was plotted against drug concentration to investigate the increase in cell proliferation.

### Real Time PCR

### RNA Extraction

HDFa cells were treated with the desired concentrations of nitrate enriched extract of Amaranth and Minoxidil for 24 hours and 48 hours. The cells were isolated after respective points and resuspended in 1 ml RNA extraction solution called Tri-reagent (Takara). The sample is kept at −20° C. for overnight and then, proceed further. 200 μl chloroform/ml of the tri reagent was added. The mixture was then vortexed and incubated at room temperature for 15 minutes followed by centrifugation at 12000 g at 2-8° C. The aqueous phase containing the RNA was transferred to a fresh tube and mixed with 0.5 ml of isopropyl alcohol/ml of tri-reagent. The mixture was incubated at room temperature for 10 minutes to ensure the complete precipitation of RNA. Then the RNA was precipitated by centrifugation at 12000 g for 10 minutes at 2-8° C. After removal of the supernatant, RNA pellet was washed once with 1 ml of 75% ethanol per ml of tri-reagent. The RNA pellet was then briefly air dried at room temperature, dissolved in Nuclease free water, and incubated at 56° C. for 10 minutes. The isolated RNA was finally quantified using Nanodrop spectrophotometer.

cDNA Preparation

Isolated RNA was given DNase treatment to remove DNA content, if any, from the sample. For this, 1U DNase and 1× DNase buffer was mixed with 1.5 μg RNA in a total of 10.0 ml reaction. The reaction was then incubated at 37° C. for 30 min followed by addition of 1 μl EDTA to stop the reaction by heating at 65° C. for 10 min. Reverse transcriptase reactions for cDNA preparation was performed by heating a 12.5 μl reaction mixture containing 1.0 μg total RNA and 0.5 μg random hexamer at 70° C. for 10 minutes. After cooling, 20 U rRNA as in ribonuclease inhibitor and 200U Moloney's murine leukaemia virus ribonuclease reverse transcriptase was added in a final 20 μl reaction mixture containing 10 mmol deoxy-NTP and 5 μl Moloney's murine leukaemia virus reaction buffer, incubated for 1 hours at 42° C. and heated 10 minutes at 70° C.

### Primer Designing and Standardization

For PCR analysis, primers for VEGF and GAPDH were designed using the Primer3 software. To ensure the specificity of selected primers, BLAST was done to align the primers with the genome sequence in the database and specificity of the sequences was checked. Primers were ordered from Eurofins. Annealing temperature of the primers was standardized by putting up gradient PCR at temperatures five degrees higher and lower the melting temperature (Tm) of the primers. A 20 μl reaction was set up.

The contents were mixed gently without forming bubbles and kept in PCR machine. This was followed by 2% agarose gel electrophoresis to check for the product formation and to select the annealing temperature giving maximum intensity of the desired product size.

### Agarose Gel Electrophoresis

The PCR products were analysed by running on 1.5% agarose gel in 1× TAE buffer. Amount of agarose corresponding to 1.5% was weighed and added to conical flask containing 1× TAE. The mixture was boiled in microwave till clear solution was obtained, allowed it to cool for some time after which Ethidium Bromide (EtBr) was added, and the gel was carefully poured on casting plate with an appropriate comb. It was allowed to solidify and then immersed in 1× TAE. The samples were loaded in respective wells and allowed to run till Bromophenol Blue reached the bottom of the gel. The gel bands were analysed in gel documentation. The temperature which shows the desired PCR product of significant intensity is chosen for real time quantitation.

### Real Time PCR

After determining the annealing temperature by Gradient PCR, Real time PCR was put for VEGF and GAPDH in all the study subjects. The real time detection of amplified PCR products is based on detection of fluorescent signals generated by binding of SYBR green to double stranded DNA. The fluorescent signal from each PCR reaction was collected as the peak-normalized values plotted vs. the cycle numbers. Reactions were characterized by comparing the threshold cycle (Ct) values. Ct is a unit-less value defined as the fractional cycle number at which the normalized sample fluorescence signal passes a fixed threshold above baseline, when it is always located within the linear phase of amplification. Samples with a high starting copy number of cDNA show an increase in fluorescence earlier in the PCR process, therefore resulting in a low Ct number. β-actin will be used to serve as internal control.

An aliquot containing total cDNA (100-200 ng) obtained from equal amount of total RNA was subjected to PCR using specific primers for VEGF and GAPDH. The PCR reactions was carried out in 10 μl total volume containing 0.3 μM of each of the primers, 1× SyBr green mix (GBiosciences). The PCR conditions comprised of 30 cycles of denaturation at 94° C. for 25 s, annealing at 60° C. for 25 s, extension at 72° C. for 25 s and fluorescence recording after extension step. GAPDH was used as an internal control for normalization.

### Western Blotting

Western blotting was performed to investigate the alteration in the protein expression of VEGF after respective treatments. β-actin was used as an endogenous control for normalization.

### Cell Lysate Preparation

Cells were harvested after respective incubation time and centrifuged at 200 g for 10 minutes to pellet down the cells. The cells were then resuspended in ˜50 μl working RIPA lysis buffer solution containing 49.5 μl of stock RIPA lysis buffer and 0.5 μl of protease inhibitor cocktail. The cells were kept on ice for 30 minutes with gentle pipetting after every 15 minutes. This was followed by centrifugation at high speed (15,000 g) for 20 minutes at 4° C. The supernatant containing cellular proteins were collected and stored in aliquots at −80° C. till further use.

### Estimation of Protein Concentration

The concentration of proteins in cell lysates was determined using Bradford assay in which 1× Bradford reagent was used for estimation and BSA (5 mg/ml) was used as standard. The stock of BSA was made in distilled water and diluted into different concentrations. The end point absorbance was read at 595 nm wavelength after 5 minutes incubation and a standard curve was plotted. The concentration of protein in unknown sample was determined using that BSA standard curve.

### Preparation of Samples for Loading Into Gels

20 μg cell lysate was mixed with 3× protein loading dye containing β-mercapto ethanol and SDS to denature the sample and impart negative charge to the protein by binding to the amino acids. The mixture was boiled at 95° C. for 10 minutes which then could be loaded into the wells of gel.

### SDS-PAGE (Sodium Dodecyl Sulphate-Polyacrylamide Gel Electrophoresis)

10-12% polyacrylamide gels (for high and low molecular weight proteins respectively) were formed from the polymerization of acrylamide and N,N-methylenebisacrylamide (or bis-acrylamide). The latter one acted as a cross-linking agent for the formation of gels. The polymerization was initiated by the addition of ammonium persulfate along with TEMED. The gels were neutral, hydrophilic, three-dimensional networks of long hydrocarbons crosslinked by methylene groups.

The samples prepared were loaded into gels and were electrophoresed at 60V and 100 V on 10-12% SDS-PAGE using 1× Tris-glycine electrophoresis buffer in Biorad western apparatus. Protein ladder (GBiosciences) was used to confirm the molecular weight of desired protein band. The movement of bromophenol blue in the loading dye marked the electrophoresis front and the gel was stopped when the dye travelled out of the gel.

### Transfer of Proteins and Staining (Western Blotting)

The contents of the gel were transferred to the nitrocellulose membrane which is more rigid and convenient for further processing. The gel and membrane were sandwiched between sponge and filter pad (sponge/filter pad/gel/membrane/filterpad/sponge) and all were clamped tightly together after ensuring no air bubbles had formed between the gel and membrane. The sandwich submerged in 1× Tris-glycine transfer buffer in Bio-Rad western transfer apparatus in such a way that gel should be towards negative electrode while membrane should be towards positive electrode. The electrical field of 80V was applied for 2 hours. The negatively-charged proteins move towards positively-charged electrode and hence, bind to the nitrocellulose membrane. Transferred proteins on membrane could be visualized by Ponceau S Red stain to assess the successful transfer of proteins which could be followed by further steps of western blotting.

### Blocking

The nitrocellulose membrane was blocked with 5% BSA for 1 hour at room temperature to prevent non-specific binding of antibodies to other part of membrane. Following blocking, membrane was washed once with 1× tris buffered saline tween (TBST).

### Primary Antibody Incubation

Membrane was then incubated with primary antibody overnight at 4° C. against VEGF and β-actin antibodies (Santa Cruz Biotechnology). Antibodies were diluted in 1% BSA/1× TBST. Followed by incubation, membrane was washed thrice with 1× TBST (10 minutes each washing).

### Secondary Antibody Incubation

The membrane was then incubated with secondary antibody anti-mouse IgG HRP conjugated (1:7000, Santa Cruz Biotechnology) for 2 hours at room temperature. The secondary antibody was diluted with 1% BSA-1× TBST. Followed by incubation, washing was done three times using 1× TBST as performed after primary antibody incubation.

### Enhanced Chemiluminescence Reaction

The membrane was developed using enhanced chemiluminescence method in which peroxide and luminol supplied in the chemiluminescence detection kit was mixed in 1:1 ratio in dark. This solution was then poured onto the membrane and incubated for around 30 seconds. The band images were acquired in Azure Biosystems followed by quantification.

### Immunohistochemistry

Paraffin blocks were cut into 5 μm thick tissue sections and mounted onto poly-L-lysine coated slides. Slides were then deparaffinized in xylene then rehydrated in alcohol down gradation. Thereafter, slides were then placed in citrate buffer (pH 6.0) 3% H₂O₂ was used to neutralize the endogenous peroxide. Primary antibody was used over tissue sections in desired dilution and incubated in 4° C. overnight. Next day, after washing HRP tagged secondary antibody was applied for 1 hour. Substrate Impact Novared was used for colour generation and was counterstained by hematoxylene. Tissue sections were then dehydrated and mounted with DPX mounting medium.

### Results

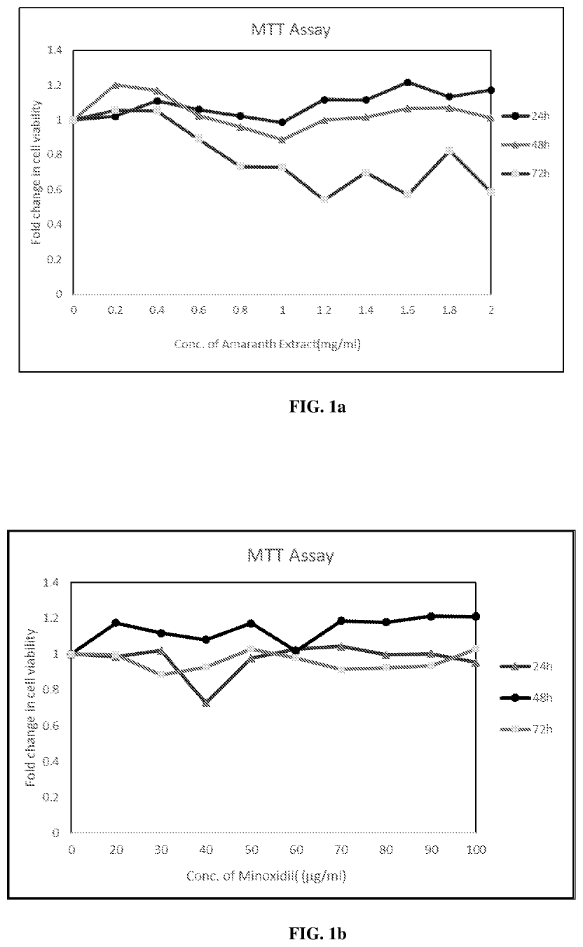

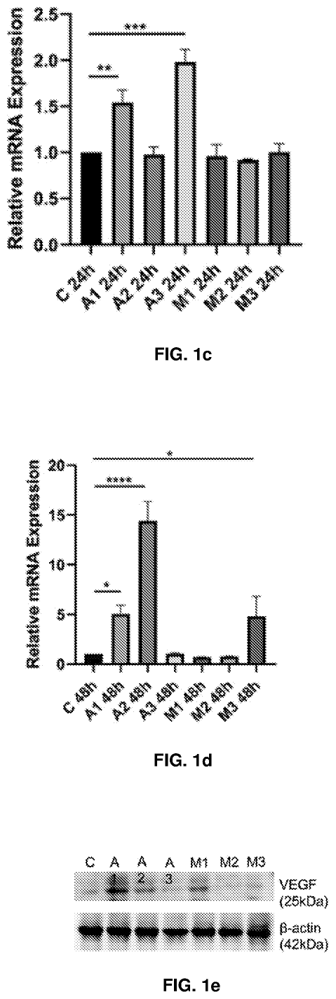

MTT Assay revealed that nitrate enriched extract of Amaranth had no side effects on the human dermal fibroblasts (HDFs). In fact, under the influence of nitrate enriched extract of Amaranth at 48 hours, HDFs exhibited ˜120 % cell proliferation. Cell cytotoxicity, however was observed at 72 hours time interval (FIGS. 1a & b). Further, in contrast to nitrate enriched extract of Amaranth, Minoxidil displayed increased cell proliferation at lower concentrations. Nonetheless, in comparison with nitrate enriched extract of Amaranth which showed its efficacy within 24 hours, Minoxidil took 48 hours to increase the rate of cell proliferation and peaked at 72 hours. It is also evident from the MTT results that nitrate enriched extract of Amaranth has a faster mode of action as compared to Minoxidil. This faster mode of action could be ascribed to the water-soluble nature of nitrate enriched extract of Amaranth, while Minoxidil is an oil-based formulation.

In vitro cell based scratch assay is one of the most apt and economic procedures for preliminary examination of wound healing potential of any medicinal extract. The current study was targeted at assessing the wound healing ability of nitrate enriched extract of Amaranth, wherein, HDFs were treated with different concentrations of nitrate enriched extract of Amaranth (0.2 mg/ml, 0.4 mg/ml, and 1.6 mg/ml) and Minoxidil (20 mg/ml, 60 mg/ml, and 100 mg/ml) for 48 hours. Cell migration was monitored at 0 hour, 24 hours and 48 hours and wound closure distance was calculated by Image J software. The scratch assay images evidently display a superior proliferative and migratory behaviour for A1 (at 0.2 mg/ml; 48 hours time period). A1 closed the gap created by the scratch by 92.142 % in 48 hours. Minoxidil on the other hand was close to the negative control (89.798 %) in its performance and closed the gap by 89.358 % at 120 mg/ml (SI 1).

VEGF mRNA expressions were examined by quantitative real-time PCR. The results clearly demonstrate that the relative mRNA expression is greater in case of nitrate enriched extract of Amaranth with A2 at 48 hours time interval being the maximum. This increased relative mRNA expression also indicates the formation of new blood vessels at the site of action/wound site. It is also important to note that the mRNA expression plumets for Minoxidil at M1 and M2 (FIGS. 1c, d& e).

The primary objective of this study however, was to investigate the modulation of VEGF protein expression in HDFs with nitrate enriched extract of Amaranth and Minoxidil. Results revealed that nitrate enriched extract of Amaranth upregulated the VEGF expression in human dermal fibroblasts; wherein Al conspicuously displayed the presence of VEGF.

VEGF is one of the most important proangiogenic molecules in the skin. It has been shown to play a role in hair growth as well as the development of skin diseases such as psoriasis and skin cancer. VEGF family members elicit their effects on endothelial cells by binding to and activating tyrosine kinase receptors located on the cell surface. VEGF is capable of binding to multiple receptors, including VEGF receptor-1 (VEGFR-1) and VEGF receptor-2 (VEGFR-2). VEGFR-2 is believed to be the more important of the two receptors in terms of controlling endothelial cell function and regulating angiogenesis based on its superior ability to stimulate downstream signaling cascades.

### Efficacy of the Topical Formulation in Male Pattern Hair Loss

Subjects of 25-45 years of age with clinically diagnosed male pattern hair loss (MPHL) with Norwood classification III vertex, IV, V were selected for the study. Subjects were divided into two groups of 25 in each group. Topical formulation of nitrate enriched extract of Amaranth is applied on vertex at a dose of 2 g once daily at night for 90 days. The primary outcome was a mean change in the density, diameter and count of Terminal hair. The secondary outcome was a mean change in the density, diameter and count of vellus, anagen, telogen, pilary index, terminal to vellus ratio, and anagen to telogen ratio.

Hair density (n/cm²)—The hair density was calculated as the number of hairs per square centimeter.

Terminal hair density (n/cm²): The number of terminal hairs per square centimeter. Terminal hair is thicker than 40 μm and this value is used by the software to identify terminal hairs in images.

Vellus hair density (n/cm²): The number of vellus hairs per square centimeter. By definition a vellus hair is thinner than 40 μm. The number of vellus hairs relative to terminal hairs is also calculated and provided in the analysis results.

Anagen hair density (n/cm²): The number of anagen hairs per square centimeter. Anagen hair grows at approximately 0.3 mm/day.

Telogen hair density (n/cm²): The number of telogen hairs per square centimeter. Telogen hairs will not grow.

Pilary Index: Percentage of anagen hairs multiplied by its diameter.

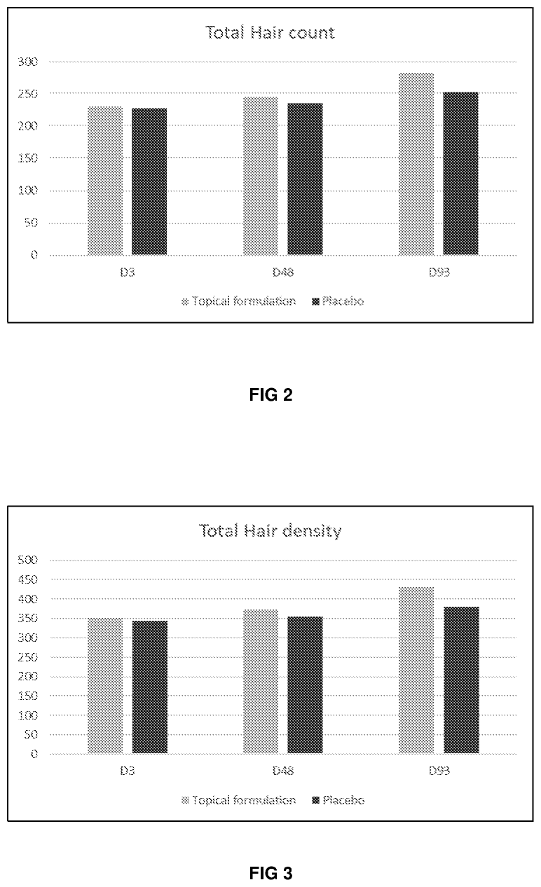

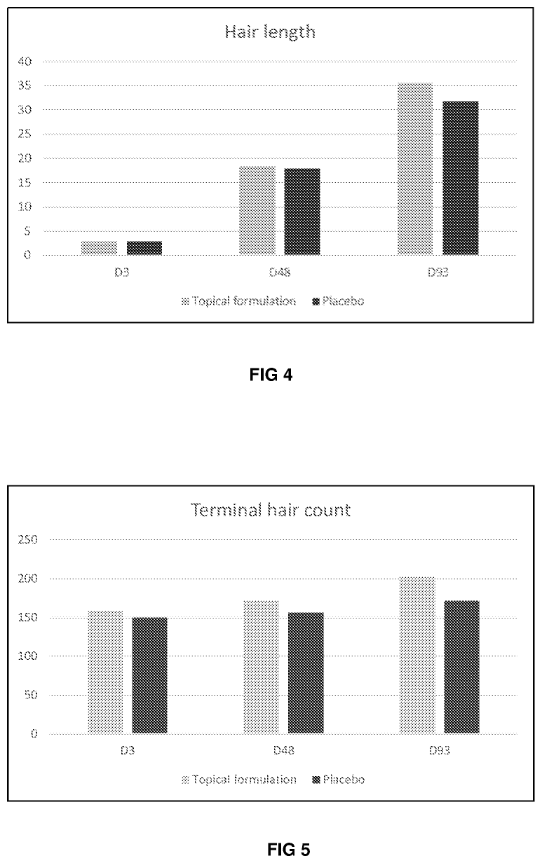

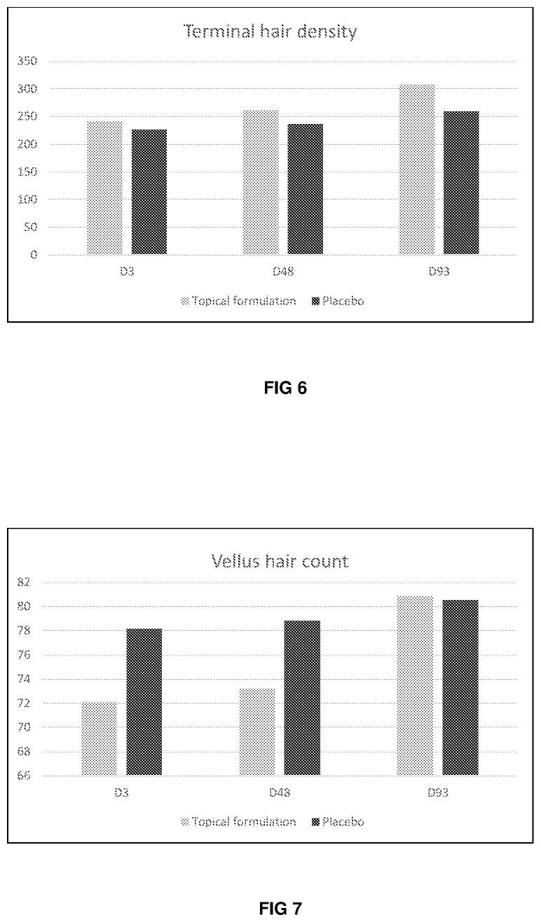

The total hair count and hair density increased significantly by 23% from baseline while placebo had 11% change (FIGS. 2 & 3) at day 90. The mean difference at day 90 was 31 and 50 respectively. The hair growth rate (μm/day) was 380 μm/day for test and 311 μm/day for placebo averaged across a study period of 90 days (FIG. 4)

The terminal hair count and density increased significantly by 28% from baseline while placebo had 15% change (FIGS. 5&6). The mean difference against placebo was 30.88 and 48.95 respectively at the end of 90 days of study. The vellus hair count and density increased by 12% in the test group whereas there was only 3% change in the placebo (FIGS. 7& 8).

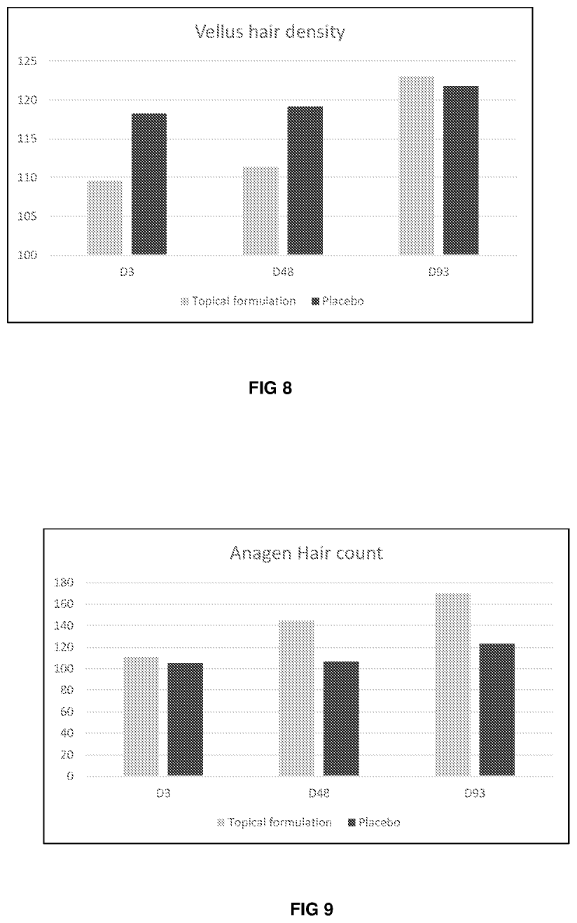

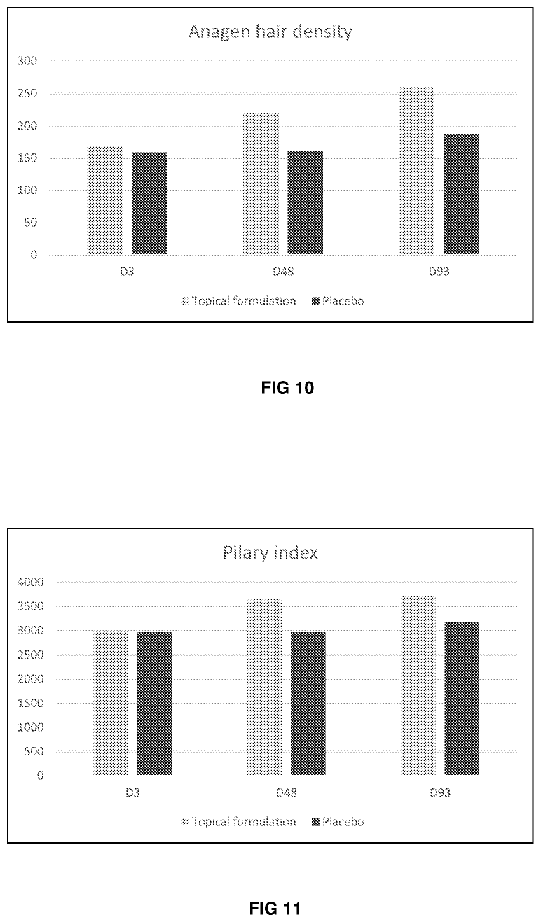

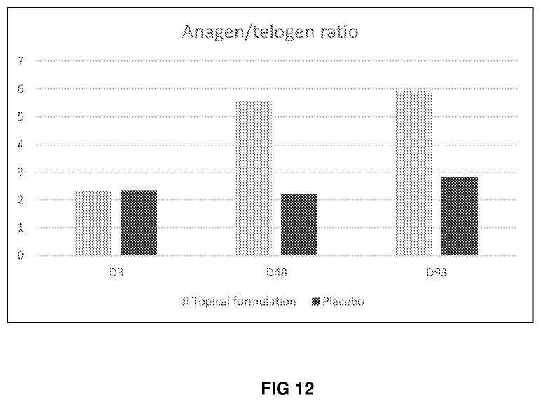

Anagen hair count and density increased significantly by 54% from baseline while placebo had 17.5% change (FIGS. 9&10). The mean difference against placebo at the end of the study was 47.08 and 73.18 respectively. The pilary index was significantly increased by 25% from baseline while placebo had 7.3% change (FIG. 11) The mean difference against placebo at the end of the study was 530.83. The anagen by telogen ratio showed significant increase of 155% whereas placebo showed only 21% change. (FIG. 12)

In this study the anagen hair density increased by 54% compared to placebo. This effect may be explained by the fact that the test formulation contains naturally occurring nitrate and the absorbed nitrate into the scalp got converted to nitric oxide increasing the blood circulation and thereby stimulating the proliferation of follicular cells.

The pilary index (PI) is a measure of the hair growth cycle in androgenetic alopecia. A lower pilary index indicates a more advanced stage of Male Pattern Hair Loss (MPHL).

In the present study the increase in PI (23%) was noticeable as early as after 45 days of application of the test product while placebo had negligible change of 0.11%. At the end of the 90^{th} day study there was a significant increase of 25% change while placebo had 7% change.

We have brought out the novel features of the invention by explaining some of the preferred embodiments under the invention, enabling a person in the art to understand and visualize our invention. It is also to understand that the invention is not limited in its application to the details set forth in the above description or illustrated in the drawings. Although the invention described in considerable detail with reference to certain preferred embodiments thereof, various modifications can be made without departing from the scope of the invention as described herein above and as defined in the following Claims.

1. A formulation for stimulating hair growth and improving quality comprising of nitrate enriched extract of Amaranth.

2. The formulation for stimulating hair growth and improving quality as claimed in claim 1, is comprising of 10-20% nitrate enriched extract of Amaranth.

3. The formulation for stimulating hair growth and improving quality as claimed in claim 1, is comprising of 15% nitrate enriched extract of Amaranth.

4. The formulation for stimulating hair growth and improving quality as claimed in claim 1, is comprising of one or more ingredients selected from liquid paraffin, mango butter, bees wax, cetyl alcohol, glycerol, Sodium Benzoate, and Potassium Sorbate.

5. The formulation for stimulating hair growth and improving quality as claimed in claim 1 is a composition of Amaranth extract comprising Amaranth nitrate and one or more ingredients selected from liquid paraffin, mango butter, bees wax, cetyl alcohol, glycerol, Sodium Benzoate, and Potassium Sorbate.

6. The formulation for stimulating hair growth and improving quality as claimed in claim 1, is having high wound healing potential at lower concentration by Increasing the rate of cell proliferation.

7. The formulation for stimulating hair growth and improving quality as claimed in claim 1, increases the hair growth rate.

8. The formulation for stimulating hair growth and improving quality as claimed in claim 1, increases total hair count and density (n/cm2).

9. The formulation for stimulating hair growth and improving quality as claimed in claim 8, increases the count and density (n/cm2) of Terminal hair.

10. The formulation for stimulating hair growth and improving quality as claimed in claim 8, increases the count and density (n/cm2) of Vellus hair.

11. The formulation for stimulating hair growth and improving quality as claimed in claim 8, increases Angen to Telogen ratio.

12. The formulation for stimulating hair growth and improving quality as claimed in claim 8, increases the count and density (n/cm2) of Anagen hair.

13. The formulation for stimulating hair growth and improving quality as claimed in claim 8, increases Terminal to Vellus ratio.

14. The formulation for stimulating hair growth and improving quality as claimed in claim 8, increases vascular endothelial growth factor (VEGF) mRNA expressions.

15. The formulation for stimulating hair growth and improving quality as claimed in claim 8, increases Pilary index which is the percentage of Anagen hairs multiplied by its diameter.

16. A method of preparation of the formulation as claimed in claim 1 for stimulating hair growth and improving quality comprising of:
combining bees wax and liquid paraffin each in an amount ranging from 20-30% and melting in a water bath to form an oil phase;
combining Cetyl alcohol and liquid paraffin each in an amount ranging from 5-10% and melting to form an oil phase;
dissolving Sodium benzoate and Potassium sorbate each in an amount ranging from 0.1-3% in water separately to form water phase;
Nitrate enriched extract of Amaranth and glycerol in an amount ranging from 10-20% and 15-25% respectively mixed with stirring to form water phase; and
adding the resultant water phase and oil phase together while stirring resulting into the formulation for stimulating hair growth and improving quality.

17. A method for stimulating hair growth and improving quality is as claimed in with formulation of claim 1 comprising applying said formulation on the temples, vertex scalp, and or mid-frontal scalp of clinically diagnosed male pattern hair loss (MPHL) at a dose of 2 g once daily at night.

18. The method of claim 17, wherein the said formulation is applied for a period of 45 days or more.
